(19) United States
(12) Patent Application Publication
(10) Pub. No.: US 2020/0327986 A1
(43) Pub. Date: Oct. 15, 2020

(54) INTEGRATED PREDICTIVE ANALYSIS APPARATUS FOR INTERACTIVE TELEHEALTH AND OPERATING METHOD THEREFOR

(71) Applicant: Samsung Electronics Co., Ltd., Suwon-si Gyeonggi-do (KR)

(72) Inventors: Agus KURNIAWAN, Jakarta (ID); Josephine KUSNADI, Jakarta (ID); Risman ADNAN, Jakarta (ID)

(21) Appl. No.: 16/756,638

(22) PCT Filed: Dec. 10, 2018
(86) PCT No.: PCT/KR2018/015610

§ 371 Date: Apr. 16, 2020

(30) Foreign Application Priority Data
Dec. 15, 2017 (ID) PID201709176

(51) Int. Cl. G16H 50/20; G16H 40/67; G16H 70/60; G16H 50/70; G16H 15/00; G16H 30/40; G06F 21/60; A61B 5/00

(52) U.S. Cl. CPC G16H 50/20; G16H 40/67; G16H 70/60; G16H 50/70; G16H 15/00; A61B 5/7264; G06F 21/602; A61B 5/0022; A61B 5/7275; A61B 5/7445; G16H 30/40

(57) ABSTRACT

The present disclosure generally relates to an integrated predictive analytics system and method that provides an interactive telehealth diagnosis, including classification and recognition of health data, recommendation analysis for health, and cognitive telehealth visualization of health data. Provided is an integrated predictive analytics apparatus for interactive telehealth. The integrated predictive analytics apparatus includes a hub configured to receive sensor data from sensor devices and generate health data by processing the sensor data and a healthcare analytics unit configured to process and visualize health data.

### TECHNICAL FIELD

The present disclosure generally relates to an integrated predictive analytics system and method that provides an interactive telehealth diagnosis, including classification and recognition of health data, recommendation analysis for health, and cognitive telehealth visualization of health data. In the present disclosure, the interactive telehealth diagnosis provides visualized results and recommendations through a web, mobile devices, and visual reality (VR) devices, such that more interaction may be provided to users in visualization of telehealth data.

### BACKGROUND ART

Recently, a number of electronic health/medical records have become available in the market. Such a large number of electronic health/medical records require a new approach to systemize and process the electronic health/medical records and new methods for visualization. However, rapid growth of digital health records is not accompanied by the availability of qualified medical experts such as doctors and medical specialists who perform complex health analysis or provide recommendations.

Thus, a need exists for a new integrated telehealth prediction system which collects health data from various sensors, carries out predictive analysis with respect to health recommendations, and enables interactive telehealth diagnosis using cognitive health visualization through a virtual reality (VR) device.

### DESCRIPTION OF EMBODIMENTS

### Technical Problem

There is a need for a new integrated telehealth prediction system which collects health data from various sensors, carries out predictive analysis with respect to health recommendations, and enables interactive telehealth diagnosis using cognitive health visualization through a virtual reality (VR) device.

### Solution to Problem

According to an embodiment of the present disclosure, an interactive predictive analytics system for telehealth (IPAST) is suggested to provide a complete integrated system and method that obtains sensor data, analyze data, and provide health recommendations by using a virtual reality (VR) device. In addition, the integrated system analyzes various health data and provides recommendations based on insight data collection.

Aspects of the present disclosure proposed herein may include:

1) Internet of Things (IoT) hub serving as a bridge between health sensor devices and a backend server;

2) Health analytics system for analysis and prediction of health data;

3) Recommendation system for health data;

4) Health data repository server for smart health data repository;

5) Visualization of cognitive health data in a three-dimensional (3D) model; and

6) Interactive analysis and diagnosis for telehealth data (IPAST).

### Advantageous Effects of Disclosure

An integrated telehealth prediction system allowing an interactive telehealth diagnosis to be made may be provided.

### BRIEF DESCRIPTION OF DRAWINGS

To understand the present disclosure and understand how the present disclosure is actually implemented, some embodiments will be described with reference to the attached drawings.

FIG. 1 is a general schematic diagram of an interactive predictive analytics system for telehealth.

FIG. 2 is a schematic diagram of a hub.

FIG. 3 is a diagram of a network module of a hub.

FIG. 4 schematically shows a data flow of a hub.

FIG. 5 schematically shows a health analytics system (HAS).

FIG. 6 shows a data flow of a health recommendation system in a medical analytics system.

FIG. 7 schematically shows a deep learning computation engine.

FIG. 8 schematically shows a data flow for learning and testing of a deep learning computation engine.

FIG. 9 shows a data flow of a health data repository system.

FIG. 10 schematically shows a process of visualizing medical data using a template.

FIG. 11 schematically shows a high-level architecture for cognitive medical data visualization.

FIG. 12 schematically shows a process of a virtual reality (VR) application that consumes and three-dimensionally (3D) visualizes health data.

FIG. 13 shows a sample of a result of rendering 3D medical data for a specific disease template.

FIG. 14 shows a flow of a task of 3D visualization of medial image data.

FIG. 15 is a schematic diagram of an application mode.

FIG. 16 schematically shows a real-time diagnosis mode using VR devices.

FIG. 17 shows a sample scenario of a review mode for identifying information provided based on a medical predictive analysis result.

FIG. 18 shows a sample scenario of an analysis mode for viewing visualization of 3D health data.

### BEST MODE

According to an embodiment of the present disclosure, an interactive predictive analytics system for telehealth (IPAST) is suggested to provide a complete integrated system and method that obtains sensor data, analyze data, and provide health recommendations by using a virtual reality (VR) device. In addition, the integrated system analyzes various health data and provides recommendations based on insight data collection.

### Mode of Disclosure

Hereinafter, embodiments of the disclosure will be described in detail with reference to the attached drawings to allow those of ordinary skill in the art to easily carry out the embodiments. However, the disclosure may be implemented in various forms, and are not limited to the embodiments described herein. To clearly describe the present disclosure, parts that are not associated with the description have been omitted from the drawings, and throughout the specification, identical reference numerals refer to identical parts.

Some embodiments of the present disclosure may be represented by block components and various process operations. All or some of such functional blocks may be implemented by various numbers of hardware and/or software components which perform specific functions. For example, functional blocks of the present disclosure may be implemented with one or more microprocessors or circuit elements for a specific function. The functional blocks of the present disclosure may also be implemented with various programming or scripting languages. Functional blocks may be implemented as an algorithm executed in one or more processors. Furthermore, the present disclosure may employ any number of conventional techniques for electronics configuration, signal processing and/or control, data processing and the like.

Connecting lines or connecting members between elements shown in the drawings are intended to merely illustrate functional connections and/or physical or circuit connections. In an actual device, connections between elements may be indicated by replaceable or added various functional connections, physical connections, or circuit connections.

According to the present disclosure, a method of improving usefulness of an integrated telehealth system by using cognitive medical visualization is proposed. The present disclosure provides a telehealth system for both a premise diagnosis and a remote diagnosis. The present disclosure also proposes a method for an interactive predictive analytics system for telehealth (IPAST), which includes a method to obtain medical data from various sensors, classify and recognize health data, provide a recommendation system for health, and visualize health analysis, and derives cognitive medical visualization. The proposed interactive remote diagnosis may support user's task by synthesizing and integrating health data from various sources and providing visualization through a cognitive visual recognition system. The present disclosure may visualize health data into a three-dimensional (3D) form through a web, mobile and/or virtual reality (VR) devices, allowing visualization of remote medical data to be further interactive for users such as doctors and/or medical specialists.

The present disclosure is proposed considering the following 6 aspects:

1) the present disclosure has been designed to develop an integrated predictive analytics system for an interactive telehealth diagnosis;

2) a system proposed in the present disclosure is pluggable, and may be integrated with any health sensors and operate as a bridge for transmitting data to a server;

3) a system proposed in the present disclosure allows a user to customize and personalize an integrated predictive analytics system according to preference of the user;

4) a system proposed in the present disclosure implements a deep learning engine system for automatically identifying related health data in providing context-based health and disease;

5) a system proposed in the present disclosure provides a health recommendation system for doctors; and

6) a system proposed in the present disclosure provides 3D visualization for health data and user interaction through a web application, a mobile application, and a VR application.

Hereinafter, the present disclosure will be described with reference to the accompanying drawings.

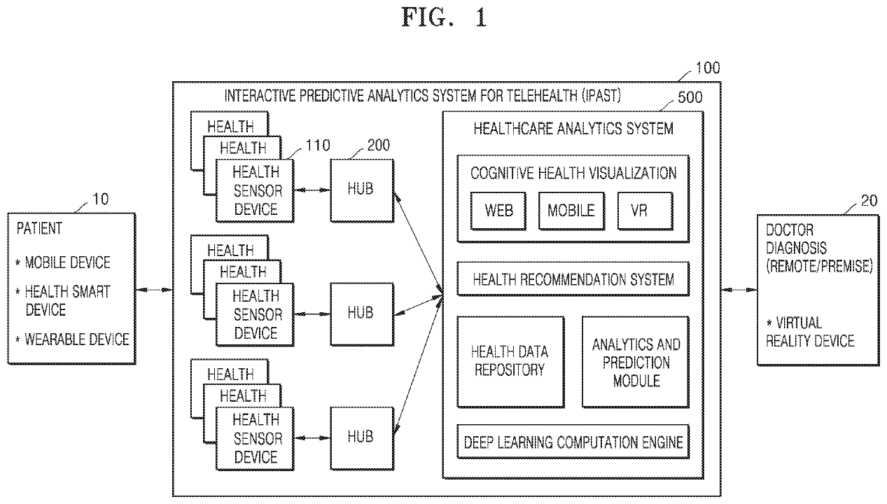

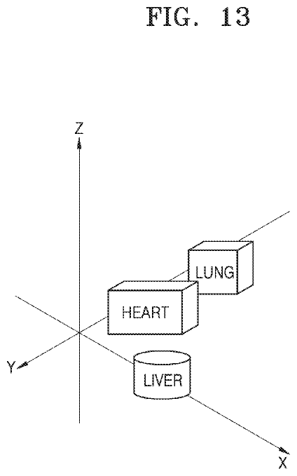

Preferred embodiments and their advantages will be understood best with reference to FIGS. 1 through 18. Thus, embodiments described herein should be understood as merely describing application of the principle of the disclosure. Mentioning about details of the embodiments described herein is not intended to limit the scope of the claims.

Referring to FIG. 1, a general schematic diagram of an interactive predictive analytics system for telehealth (IPAST) for telemedicine according to an embodiment of the present disclosure is shown.

As shown in FIG. 1, an IPAST 100 includes two systems as below.

A hub 200 is a network device that provides connection between health sensor devices 110 and a server. For example, the hub 200 may be an embedded system of the IPAST 100. The hub 200 may perform connection between health sensor devices 110 and a healthcare analytics system (HAS) 500 or connection between components of the IPAST 100 and a network.

The HAS 500 may be a core system of the IPAST 100, which includes several modules. The HAS 500 may be included in or include a server or may include or be included in an electronic device. Modules of the HAS 500 shown in FIG. 1 may be included in different electronic devices or servers.

The hub 200 may perform connection between the health sensor devices 110 and the HAS 500. The hub 200 may perform protocol form conversion and minimum routing with respect to a target server. A health measurement result from the health sensor devices 110 may be transmitted to the HAS 500. The health sensor devices 110 may have different protocol forms for transmitting sensor data, such that the HAS 500 complies with a protocol form.

The IPAST 100 may be designed to achieve the following goals of:

1) utilizing a large volume of medical records stored in the IPAST 100, supporting clinical decision, and improving the quality of treatment;

2) designing and developing a scalable deep learning computation system supporting various deep learning models, techniques, and algorithms to perform various predictive analyses in a medical field such as clinical images, electronic health records (EHR), and mobile sensing (e.g., mobile sensing using smartphones, wearable devices, etc.);

3) modeling a medical process between a doctor 20 and a patient 10 in the IPAST 100 by using a Markov decision process (MDP) having an appropriate compensation function from the doctor, in which a compensation value is not included in medical records collected from a sensor; and

4) interfering an optimal action selection rule (or policy) according to a patient's state based on a doctor's previous treatment material. In general, a doctor makes a decision based on a personal opinion, but the IPAST 100 reuses an expert's experience to analyze health and provide related recommendations.

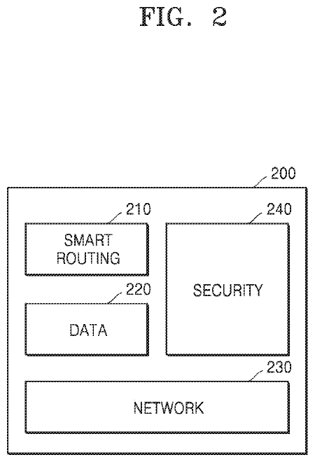

Referring to FIG. 2, a schematic diagram of a hub design according to an embodiment of the present disclosure is shown. As shown in FIG. 2, a hub 200 may serve as a bridge between the health sensor devices 110.

Each sensor 110 may be connected to a particular embedded board to capture and measure a physical object in a digital form. Upon completion of a detection process, sensor data may be delivered to a target system. The hub 200 may be an end-point sever for all the health sensor devices 110. The hub 200 may also provide a smart routing module 210 for routing the sensor data to a particular target sensor. The hub 200 may perform protocol form conversion from a system form to another system form. The hub 200 may include various components enabling data exchange between the health sensor devices 110 and a target system.

As shown in FIG. 2, the hub 200 may include different components. Each component of the hub 200 may operate a system well and will be briefly described below.

The smart routing module 210 may provide a function of routing data to an appropriate target and guiding a path, by using the shortest path and a narrow bandwidth. The smart routing module 210 may control data communication between entities based on applied optimized routing and bandwidth usage.

A data module 220 may control encoding and decoding based on a protocol of the data module 220. The data module 220 may operate as a cache server for storing data and providing available functions.

A network module 230 may manage all transmission/reception data and address heterogeneous protocols. The network module 230 may monitor incoming and outgoing data and serve as a bridge for translation from one protocol scheme to another protocol scheme.

A security module 240 may guarantee secure communication between the hubs 200. The security module 240 may guarantee that all health data is secure. The security module 240 may apply encryption computation to data to provide integrity of data.

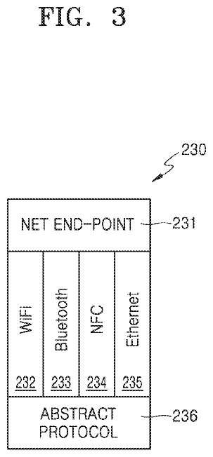

Referring to FIG. 3, a schematic diagram of a design of the network module 230 according to an embodiment of the present disclosure is shown. As shown in FIG. 3, network components on the hub 200 are designed to enable communication through heterogeneous protocols. The network module 230 of the hub 200 may provide various common protocol stacks to enable all communications. The network module 230 may include components as below.

A net end-point module 231 may be an interface capable of communicating with another system through a particular protocol. The net end-point module 231 may have a network interface-based protocol form. The net end-point module 231 may provide various standard protocols such as Wireless Fidelity (Wi-Fi) 232, Bluetooth 233, near field communication 234, and Ethernet 235 to communicate with another system through different types of protocols.

An abstract protocol 236 may implement a generic protocol to be used by a system to process a next process. The abstract protocol 236 may be a version of generalization of a protocol such that the hub 200 supports various requests/responses from the net end-point module 231.

The hub 200 may support various data protocols, and convert data with an appropriate protocol in a remote medical system. The hub 200 may implement a cross-layer access to optimize data communication between the hub 200 and the health sensor devices 110.

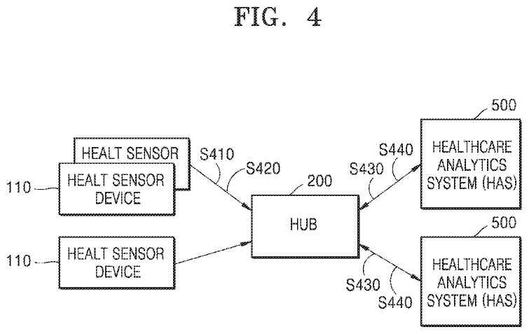

Referring to FIG. 4, a schematic diagram of a data flow on the hub 200 according to an embodiment of the present disclosure is shown.

As shown in FIG. 4, the data flow on the hub 200 may be described with operations as below.

In operation S410, the third-party health sensor devices 110 may perform sensing to obtain health data. The sensed data may be transmitted to the hub 200 through a sensor protocol. The health sensor device 110 may perform sensing at a particular time and transmit the sensed data to the hub 200 by using a unique network protocol.

The hub 200 may include several protocol end-points capable of communicating with all the sensor devices 110. In operation S420, the hub 200 may open all network interfaces and wait for incoming sensor data.

In operation S430, the hub 200 may perform prior processing including protocol message translation. The health sensor devices 110 and a target server 500 have their self-protocol forms, such that the hub 200 may convert a sensor device protocol into a target server protocol. After reception of sensor data, the hub 200 may parse data and reconstruct data according to a data server form.

The hub 200 may perform smart routing allowing connection to a target server with a minimum effort. The hub 200 may perform bandwidth optimization computation to transmit data to a server.

In operation S440, the hub 200 may transmit data. The HAS 500 may be a server target of the hub 200. The hub 200 may send sensor data to the HAS 500. The HAS 500 may perform computation based on received data. After reception of data, the HAS 500 may perform data analysis to provide health recommendations.

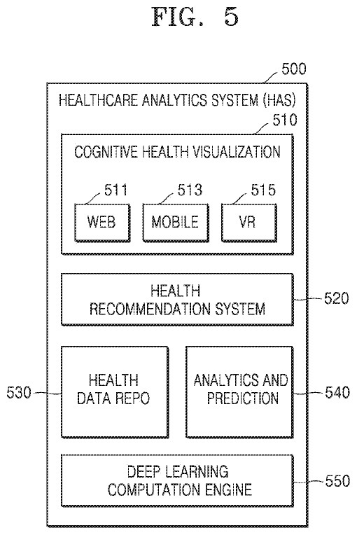

Referring to FIG. 5, a schematic diagram of the HAS 500 according to an embodiment of the present disclosure is shown.

As shown in FIG. 5, the HAS 500 may be a core system of the IPAST 100 including several components for manipulating an analytics system and computation. A list of the components included in the HAS 500 of the IPAST 100 is shown in FIG. 5. The HAS 500 may operate in a single server or a multi-server of a farm environment. The HAS 500 may be arranged in several positions to provide a service to all entities. The HAS 500 may analyze remote digital images such as magnetic resonance imaging (MRI) images and an image including information about an overall health state of a patient (e.g., a temperature, etc.). The HAS 500 may include an algorithm for classifying a type of remote medical images. The HAS 500 may analyze a remote medical image by using machine learning.

More specifically, the HAS 500 may include modules and/or functions described below.

A health recommendation system module 520 may perform computation for generating recommendations about health behavior based on input data. The health recommendation system module 520 may use an analytics and prediction module in performing computation. The health recommendation system module 520 may provide health recommendations and proposals based on health data for a particular purpose.

A health data repository (health data repo) module 530 may be a health data repository having various health data types such as diseases.

The cognitive health visualization module 510 may be an engine for visualizing health data on a web platform 511, a mobile platform 513, and a VR platform 515. The cognitive health visualization module 510 may generate 3D images through region segmentation, 3D depth prediction, plane estimation, etc.

The analytics and prediction module 540 may be a module for analyzing and predicting particular data. In a computing process, the analytics and prediction module 540 may use a deep learning computation engine module 550. The analytics and prediction module 540 may analyze collected health data and generate data insight.

The deep learning computation engine module 550 may be an engine system that performs deep learning computation for a particular purpose. The deep learning computation engine module 550 may be used by any module of the HAS 500. Technically, the deep learning computation engine module 550 may use a deep learning algorithm as a core computation scheme. For example, the deep learning computation engine module 550 may implement reinforcement learning to compute health data.

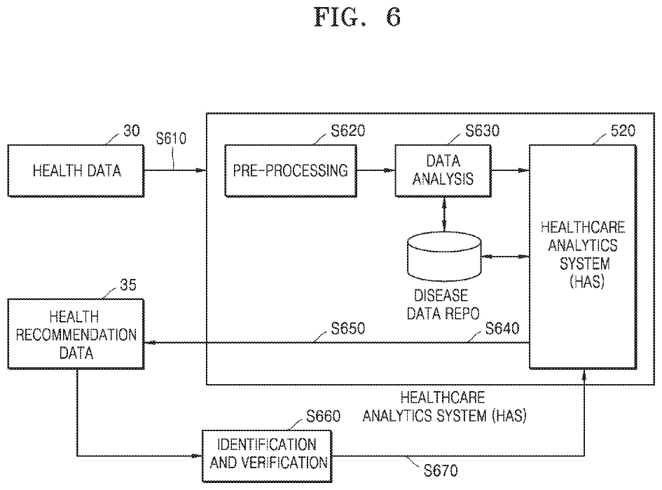

Referring to FIG. 6, a data flow of a health recommendation system 520 on the HAS 500 according to an embodiment of the present disclosure is shown. As shown in FIG. 6, the health recommendation system 520 is one of features of the HAS 500. The health recommendation system 520 may collect user health input data and data of a local health repository and perform deep learning computation to generate several recommendations related to health measures.

The health recommendation system 520 may provide health recommendations based on one disease type or a plurality of disease types. When the health data includes a single disease type, the health recommendation system 520 may provide particular health recommendations corresponding to disease characteristics. When the health data includes several disease types, the health recommendation system 520 may aggregate various health recommendations suitable for health data and provide at least one health recommendation.

The following operations describe a data flow in the HAS 500 according to an embodiment.

In operation S610, health data 30 may be transmitted to the HAS 500 by a hub 200 by using data in a particular form and a network protocol.

In operation S620, all the health data 30 arriving at the HAS 500 may undergo prior processing for address identification and data verification. The prior processing in operation S620 may be useful to prepare for data for analysis.

In operation S630, the HAS 500 may analyze obtained and/or collected data to obtain disease insight data by using internal disease/illness data through a data analysis process.

In operation S640, the health recommendation system module 520 may perform particular computation to generate health recommendations based on disease recognition data. Upon completion of computation, in operation S650, a recommendation result 35 may be transmitted to a user or a requester.

In operation S660, a user such as a doctor may evaluate, identify, and reject the recommendation result 35 of the system. A rejection result may be transmitted to the server 500 for an additional evaluation and learning process. In operation S670, a feedback from the user may be submitted to the server for reference by the server.

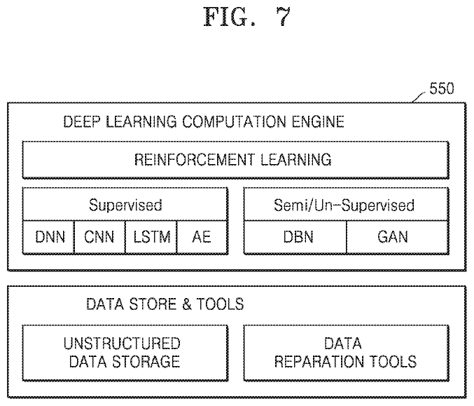

Referring to FIG. 7, a schematic diagram of the deep learning computation engine 550 according to an embodiment of the present disclosure is shown. As shown in FIG. 7, the deep learning computation engine 550 is a deep learning software library that performs much medical prediction by supporting various models and algorithms.

The deep learning computation engine 550 is a system for establishing various deep neural networks models such as a deep feed-forward neural network (DNN), a convolutional neural network (CNN), an auto encoder (AE), and a recurrent neural network (RNN) on top of stateful dataflow graphs representation. The deep learning computation engine 550 may implement various parallelism techniques on several central processing units (CPUs) and graphics processing units (GPUs).

Technically, the deep learning computation engine 550 may support the following deep neural networks models and algorithms:

1) reinforcement learning
Bayesian inverse reinforcement learning (B-IRL), and
deep inverse reinforcement learning (D-IRL);

2) supervised models
deep feed-forward neural network (DNN),
convolutional neural network (CNN),
long short-term memory (LSTM), and
auto encoder (AE); and

3) semi/un-supervised models
deep belief network (DBN), and
generative adversarial network (GAN).

The deep learning computation engine 550 may provide a framework and a tool for performing prior processing and predictive analysis with respect to collected data in the IPAST 100. The IPAST 100 may support various types of data including electronic health records, images, sensor data, and texts. Such various types of data is complex, is of different kinds, is wrongly annotated, and is generally unstructured. Data preparation tools correspond to a software library that performs various tasks related to data search and manipulation for deep learning computation. In the IPAST 100, deep learning computation is used to learn data by using a predefined model and to perform inference computation for prediction. The IPAST 100 may support a supervised model, a semi/un-supervised model, and a deep learning model classified as a reinforcement paradigm.

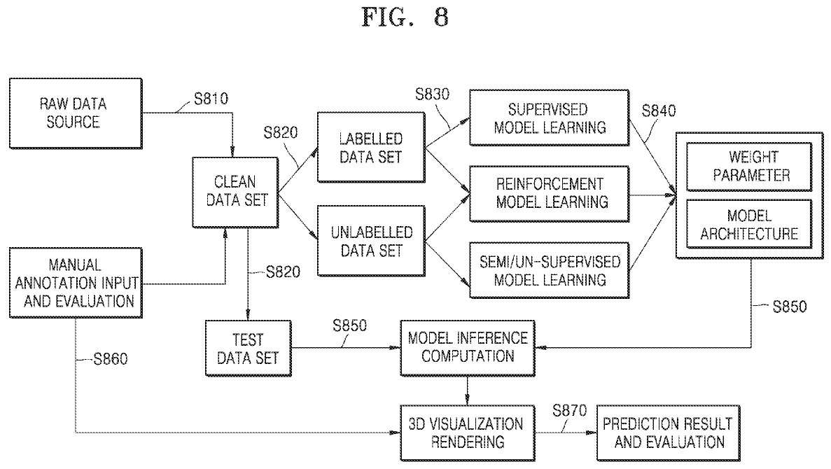

Referring to FIG. 8, a schematic diagram of learning and test with respect to the deep learning computation engine 550 according to an embodiment of the present disclosure is shown. As shown in FIG. 8, a training and test process for the deep learning computation engine 550 may include operations provided below.

In operation S810, the deep learning computation engine 550 may perform data pre-processing. Data pre-processing is intended to convert and manipulate a raw data source or a non-processed data source into a clean data set. In operation S810, the IPAST 100 may provide more context information to raw data by permitting annotation manually added by the user and dynamic evaluation.

In operation S820, the deep learning computation engine 550 may prepare for deep learning data. In an operation of deep learning data preparation using the clean data set, the deep learning computation engine 550 may need to separate data for training and test. A result of operation S820 is a data set for model learning, and may be classified into a labelled data set, an unlabelled data set, and a test data set.

In operation S830, the deep learning computation engine 550 may provide a deep learning model. The IPAST 100 may provide a deep learning model library capable of performing various prediction learning tasks such as classification and clustering. A labelled data set may be an input for supervised model learning and reinforcement model learning for classification. A non-labelled data set may be an input for data clustering using semi/un-supervised model learning.

In operation S840, the deep learning computation engine 550 may perform deep learning. An optimal result for a deep learning lifecycle may be weight parameters and an architecture of a model (e.g., a neural network topology, a hyper parameter, etc.). The deep learning lifecycle may not be a simple process. First, the deep learning lifecycle may need initial setting of a hyper parameter, such as an activation function, weight initialization, normalization, and gradient descent optimization. Second, the deep learning lifecycle may require continuous monitoring and evaluation for dynamic learning.

In operation S850, the deep learning computation engine 500 may perform model inference computation. The model inference computation is a main operation of predictive analysis. An input of operation S850 may be weight parameters and an architecture of a model. The input of operation S850 is used to perform inference computation based on a given testing data set or new data input from a patient.

In operation S860, the deep learning computation engine 500 may support manual annotation input and evaluation. The IPAST 100 may support additional and evaluation of annotation by a human to obtain a clean input data set by using more context information. In reinforcement learning, labelled data and unlabelled data may be used to train a model using a human agent who performs additional and evaluation of annotation for a label.

In operation S870, the deep learning computation engine 500 may perform model evaluation. The IPAST 100 may provide various methods and techniques for performing model evaluation based on an inference computation result for a particular learning model. Together with evaluation by the human, a prediction result and evaluation may be used to correct the entire data and a model.

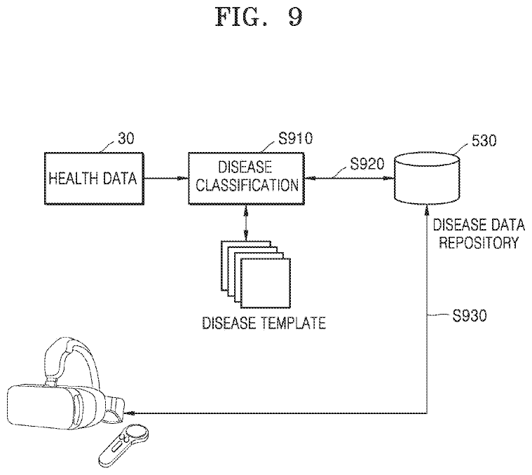

Referring to FIG. 9, a data flow of the health data repository module 530 according to an embodiment of the present disclosure is shown. As shown in FIG. 9, each health data 30 submitted to the HAS 500 may be stored in the health data repository module 530. The health data repository module 530 may be designed to manage all state data according to a type of data. Each health data 30 may be intelligently matched to a health template. The health data of the health data repository module 530 may be used as training data of the deep learning computation engine 550.

For many disease/illness types, the health data repository module 530 may provide a disease template for each disease/illness type. The health data repository module 530 may apply a dynamic data model to process various disease type data. In order for a cognitive health data visualization engine to render two-dimensional (2D)/3D state data based on a template, the health data repository module 530 may be used. As shown in FIG. 9, a data flow for the health data repository module 530 may include operations provided below.

First, the newly submitted health data 30 may be pre-processed so as to be processed in the next operation.

In operation S910, the newly submitted health data 30 may be analyzed and classified according to disease classification. In operation S910, the deep learning computation engine 550 may be engaged. By comparing the newly submitted health data 30 with a previously existing disease template, classification based on machine learning for newly collected health data may be performed.

When the type of the newly submitted health data 30 is identified, the data may be stored in the health data repository module 530 in operation S920. When the type of the newly submitted health data 30 is not identified, the data may be rejected.

In operation S930, a doctor may verify an identification process and determine whether classification is correct. The doctor may reject a result of a classification process of operation S910. The HAS 500 may perform health data visualization. For health data visualization, a VR device may be used.

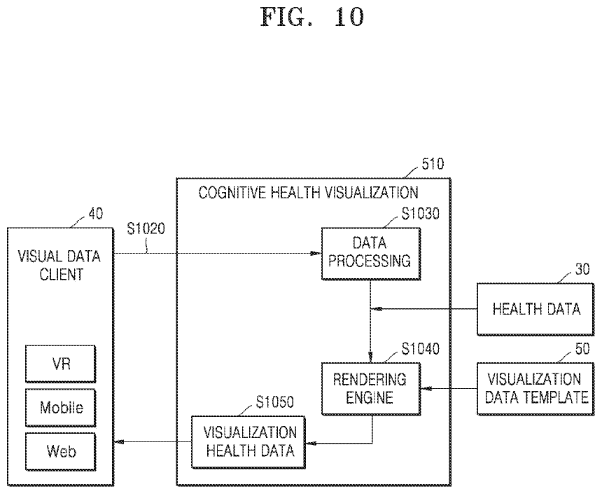

Referring to FIG. 10, a schematic diagram of a process of visualizing health data by using a template by the HAS 500 according to an embodiment of the present disclosure is shown. As shown in FIG. 10, a process of visualizing the health data 30 is shown. A disease/illness template refers to health data of a template. The health data 30 may have a unique template, and thus the health data 30 may be rendered with other persons from different points of view in visualization. As shown in FIG. 10, a process of visualizing health data in which health information is combined with a template may include operations provided below.

In operation S1020, a visual data client 40 may be expressed as a web/mobile/VR application that requests visualization of health data for a particular disease/illness. The visual data client 40 may request the cognitive health visualization module 510 to visualize health data.

The HAS 500 according to an embodiment of the present disclosure may provide visualized health data to a user through a separate device by communicating with a separate device in which the visual data client 40 is stored and installed. Alternatively, the HAS 500 may include the visual data client 40 and may provide visualized health data to the user through a display.

In operation S1030, the cognitive health data visualization engine 510 may process a request of the visual data client 40.

In operation S1040, the cognitive health data visualization engine 510 may render the health data 30 and a visualization related template 50.

In operation S1050, a result of rendered health data may be transmitted to the visual data client 40. The visual data client 40 such as the web/mobile/VR application may show the health data in the form of a 3D model. Each health data may be visualized in another form based on a template of the health data.

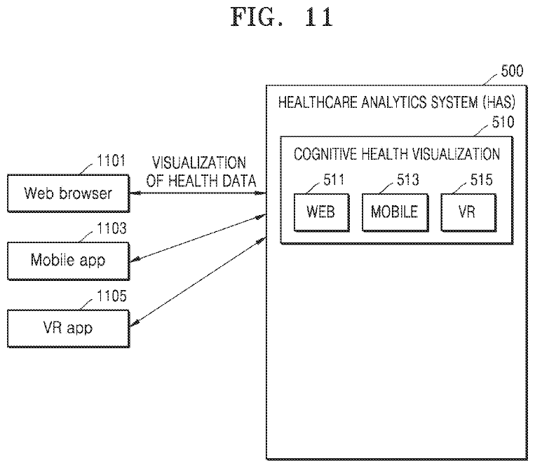

Referring to FIG. 11, a schematic diagram of a high-level architecture for cognitive health data visualization according to an embodiment of the present disclosure is shown. As shown in FIG. 11, health data may be expressed in a visual form for targeting a web browser 1101, a mobile app 1103, and a VR app 1105. The HAS 500 may provide smart data visualization including user interaction for a 3D model and health. A purpose of health data visualization may provide detailed information and data to a visual model to facilitate analyses by a doctor through accurate measurement.

The HAS 500 may include a display unit for visualizing health data and displaying the health data to the user or displaying a user interface. The HAS 500 may be connected with an external device such as a mobile device to display visualized health data. The HAS 500 may be included in a device including a display unit to display health data.

Health data visualization on the web browser 1101 may not require a special app. The health data visualization on the web browser 1101 may use visual 3D data, multimedia, and optimized hypertext markup language (HTML)5 enabling user interaction. The user may use a browser indicating an address of the HAS 500. The user may use a web browser using the address of the HAS 500 and may be provided with the visualized health data.

Meanwhile, the mobile app 1103 used to obtain health data visualization needs to include a special app established in a general operating system such as Android and/or iOS to use data of the HAS 500. The mobile app 1103 may intelligently implement 3D visualization for health data and enable user interaction. On top of the mobile app 1103, a VR app 1105) allowing the user to analyze health data through more interaction by rendering the health data in a 3D model/form may be designed.

The VR app 1105 may be designed to allow the user to further interact with the system. As the health data is provided by being rendered in a 3D form, users such as a doctor may interact with the health data.

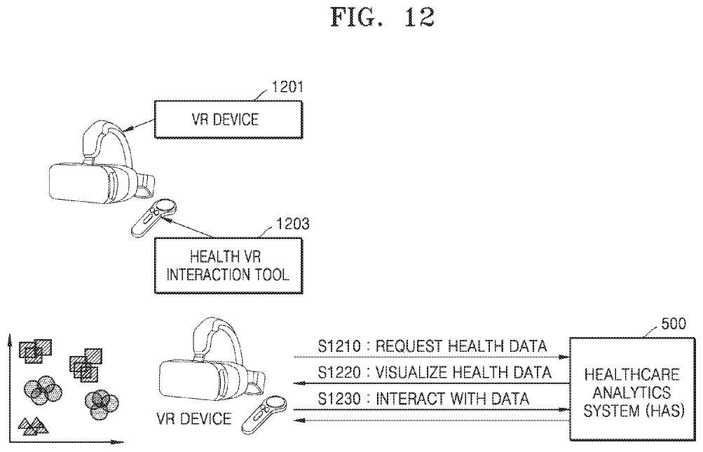

Referring to FIG. 12, a schematic diagram of a process of a VR app for consuming health data and 3D visualizing the health data according to an embodiment of the present disclosure is shown. Meanwhile, a rendering result sample of 3D health data for a particular disease template according to an embodiment of the present disclosure is shown. A method in which the VR app processes the obtained health data may include operations as below. All health data may be visualized as a 3D model by using VR devices, facilitating interaction and manipulation for data.

A VR device 1201 may be used to visualize health data. A health VR interaction tool 1203 may be used to perform interaction for the health data.

In operation S1210, a client app of the VR device 1201 such as a browser, a mobile app, or a VR app may transmit a request for state information to the HAS 500. To allow the health data to be accessed by an appropriate user, a security problem may occur. Thus, in operation S1210, it may be verified whether the user requesting data is the appropriate user.

In operation S1220, the client app of the VR device 1201 may perform 3D visual rendering from the health data after the client app obtains the health data from the HAS 500.

Upon completion of health data rendering, the client app of the VR device 1201 may interact with users in operation S1230.

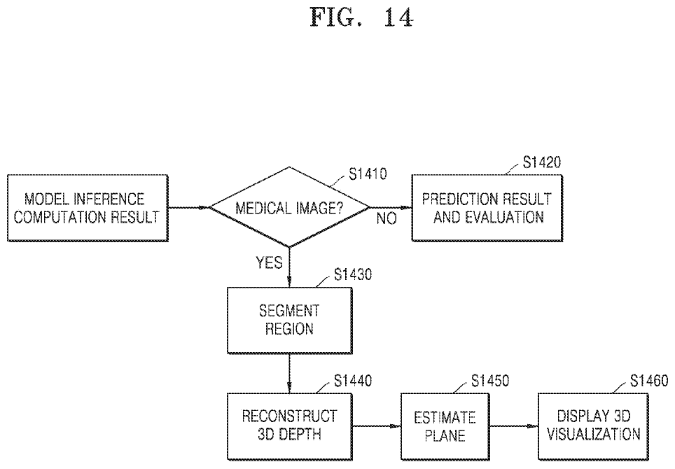

Referring to FIG. 14, a flowchart of 3D visualization of health image data according to an embodiment of the present disclosure is shown. As shown in FIG. 14, 3D visualization of health image data may be performed from a health data computation result. The cognitive health visualization module 510 may perform visual representation of electronic health data according to operations as below.

In operation S1410, the cognitive health visualization module 510 identify whether the health data computation result is associated with a medical image. When the health data computation result is not associated with the medical image, a prediction result or an evaluation result may be displayed.

On the other hand, when the health data computation result is associated with a 2D medical image such as an optical image or an MRI result indicating a skin disease, the cognitive health visualization module 510 may need to perform region segmentation in operation S1430. Region segmentation may use spatial representation for visualization of a human anatomical structure extracted from a 2D image.

In operation S1440, the cognitive health visualization module 510 may perform 3D depth reconstruction of a human anatomical structure through surface rendering and volume rendering of a 2D imaging data set such as texture-based volume rendering.

In operation S1450, the cognitive health visualization module 510 may perform 3D plane estimation. 3D plane estimation may allow multiple views of a 3D imaging result such as angle emphasis.

In operation S1460, the cognitive health visualization module 510 may display 3D visualization.

A process described in relation to health data visualization is intended to provide a medical insight for a graphic element used to deliver medical data and intuitively review a result. However, an embodiment of the present disclosure is not limited to the foregoing description. Hereinbelow, a description will be made of an example showing a way for a user to use the IPAST 100 by using various devices such as a smartphone and a VR device.

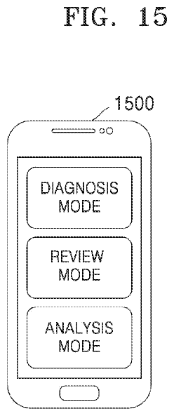

Referring to FIG. 15 showing an example of an operation method of the IPAST 100, an overview of an app mode according to an embodiment of the present disclosure is shown.

The IPAST 100 may include a display unit for visualizing medical data and displaying the medical data to the user or displaying a user interface. The IPAST 100 may be connected with a mobile device 1500 shown in FIG. 15 to display visualized medical data. Alternatively, the IPAST 100 may be included in the mobile device 1500 including the display unit and support telehealth.

As shown in FIG. 15, by using interactive data visualization, a doctor may not only view 2D medical image data, but also perform various tasks. Various tasks using interactive data visualization may include searching for medial image data in a 3D form to provide better analysis and verifying a health prediction analysis result through reinforcement learning characteristics.

A user interface for cognitive health visualization may include software installed in a mobile device such that a user like a doctor performs main tasks described below.

1) Real-time diagnosis mode using a head mounted device or a VR device

2) Review mode for obtaining telehealth information of a patient and verifying a predictive analysis result

3) Analysis mode for performing 3D health data visualization

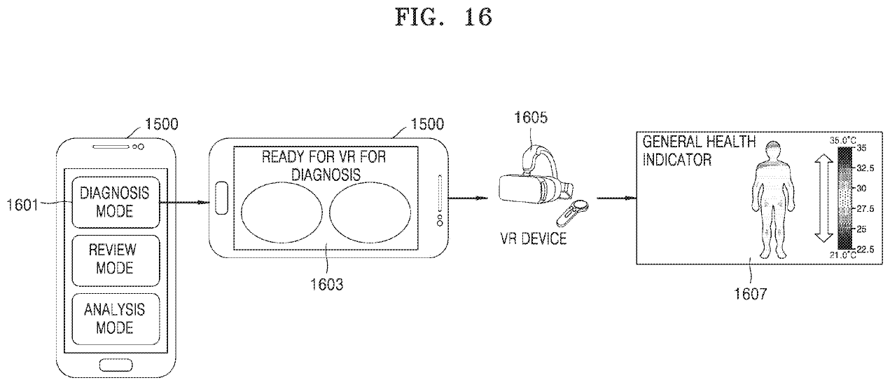

Referring to FIG. 16, a schematic diagram of the real-time diagnosis mode using the VR device according to an embodiment of the present disclosure is shown. First, a user may input a command to select a real-time diagnosis mode 1601 to the mobile device 1500. The mobile device 1500 may display a screen 1603 indicating that a VR image for a diagnosis is ready. Referring to FIG. 16, when the mobile device 1500 is paired with additional peripherals 1605 such as an infrared (IR) imaging camera and a head mounted device, a function of visualizing an overall health state of the patient by using a thermal imaging technique may be available to the doctor. The doctor may test the overall health state of the patient based on a provided image 1607 and store an image 1607 in a profile of the patient and store the same in a database, thus reviewing and analyzing information in more detail.

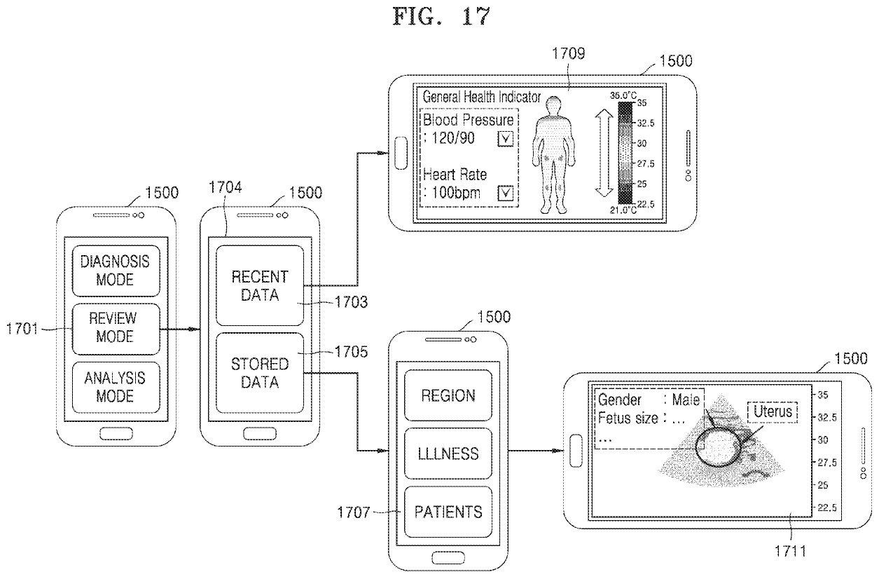

Referring to FIG. 17, according to an embodiment of the present disclosure, a sample scenario of a review mode 1701 for verifying information provided by a health predictive analysis result is shown. As shown in FIG. 17, when the mobile device 1500 is used, the doctor may verify health information provided by the health predictive analysis result by inputting the command to select the review mode 1701. Health information verified in the verification mode (human in loop verification) may include a biometric signal, heat/temperature, entire waveform electrocardiogram (ECG), etc.

As shown in FIG. 17, the doctor may verify the health diagnosis result of the patient and perform an essential change ensuring validity of provided data. As shown in a screen 1704 of FIG. 17, the mobile device 1500 may provide an option 1703 for reviewing directly obtained recent data of the patient and an option 1705 for reviewing data of another patient, stored in the database. To guarantee a privacy of health information of the patient, only an authorized doctor may review data.

As shown in FIG. 17, the mobile device 1500 may allow the user to add an annotation by handwriting to the health image data. When the IPAST 100 automatically detects connection of the mobile device 1500 to the VR device and/or the doctor selects an “analysis mode”, the IPAST 100 (or the mobile device 1500) may provide 3D image visualization to the user.

After the doctor performs a diagnosis by using the VR device in reviewing the recent data, the doctor may directly switch from the “diagnosis mode” to the “review mode”, thus capturing and reviewing an image through a mobile device such as a smartphone, a tablet, or other devices. As shown on a screen 1709 of FIG. 17, the mobile device 1500 may provide a directly obtained recent medical image of the patient.

When the doctor reviews stored data, the doctor may review and verify data of the patient stored in a telehealth database. An authorized doctor may search for and obtain data of the patient. The data of the patient may be searched for based on a particular criterion (e.g., a region, a disease type, a patient's name, etc.). As shown in FIG. 17, the mobile device 1500 may provide the screen 1707 for selecting a criterion of data search and search for data of the patient according to the selected criterion based on a user input. As shown on a screen 1711 of FIG. 17, the mobile device 1500 may provide a previously stored medical image of the patient.

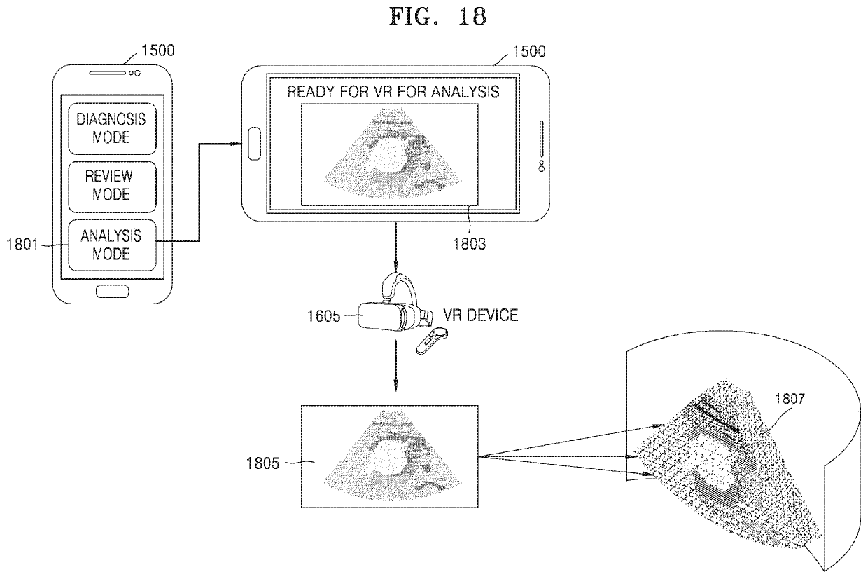

Referring to FIG. 18, a sample scenario of an analysis mode for providing 3D health data visualization according to an embodiment of the present disclosure is shown. As shown in FIG. 18, in the analysis mode, the doctor may perform visualization health analysis from health data previously stored in the IPAST 100. First, the user may input a command to select an analysis mode 1801 to the mobile device 1500. The mobile device 1500 may display a screen 1803 indicating that a VR image for analysis is ready. The IPAST 100 may provide 3D health data visualization to the user by pairing the mobile device 1500 with the VR device 1605. The IPAST 100 may perform region segmentation, 3D depth reconfiguration, and plane estimation to display a 2D image 1805 as a 3D image 1807.

Disclosed embodiments may be implemented as a software (S/W) program including an instruction stored in a computer-readable storage media.

The computer may invoke stored instructions from the storage medium and operate based on the invoked instructions according to the disclosed embodiment, and may include an image transmission device and an image reception device according to the disclosed embodiments.

The computer-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

The electronic device or the method according to the embodiments of the disclosure may be included and provided in a computer program product. The computer program product may be traded as a product between a seller and a buyer.

The computer program product may include a software (S/W) program and a non-transitory computer-readable recording medium in which the S/W program is stored. For example, the computer program product may include a product (e.g., a downloadable application) in the form of a S/W program electronically distributed through a manufacturer or the electronic device or an electronic market. For the electronic distribution, at least a portion of the S/W program may be stored in a storage medium or temporarily generated. In this case, the storage medium may be a storage medium of a server in the manufacturer or the electronic market or a relay server. In this case, the storage medium may be a storage medium of a server in the manufacturer or the electronic market or a relay server that temporarily stores the S/W program.

The computer program product may include a storage medium of a server or a storage medium of a terminal (e.g., a backend server and a device), in a system including the server and the terminal. Alternatively, when there is a third device (e.g., a smart phone) communicating with the server or the terminal, the computer program product may include a storage medium of the third device. Alternatively, the computer program product may include a S/W program itself, which is transmitted from the server to the terminal or the third device or transmitted from the third device to the terminal.

In this case, one of the server, the terminal, and the third device may execute the computer program product to perform the method according to the embodiments of the disclosure. Alternatively, two or more of the server, the terminal, and the third device may execute the computer program product to execute the method according to the embodiments of the disclosure in a distributed manner

For example, a server (e.g., a cloud server or AI server, etc.) may execute a computer program product stored in the server to control the terminal communicating with the server to perform the method according to the embodiments of the disclosure.

In another example, the third device may execute the computer program product to control the terminal communicated with the third device to perform the method according the disclosed embodiment. More specifically, the third device may remotely control the image transmission device or the image reception device to transmit or receive a packing image.

When the third device executes the computer program product, the third device may download the computer program product and execute the downloaded computer program product. Alternatively, the third device may execute a computer program product provided in a preloaded state to execute the method according to the disclosed embodiments.

1. An integrated predictive analytics apparatus for interactive telehealth, the integrated predictive analytics apparatus comprising:
a health data repository storing disease data; and
a healthcare analytics unit configured to process and visualize, based on the disease data, health data generated based on sensor data obtained from sensor devices, the healthcare analytics unit comprising a computation engine.

2. The integrated predictive analytics apparatus of claim 1, further comprising a hub configured to receive the sensor data from the sensor devices and generate the health data by processing the sensor data,
wherein the hub comprises:
a smart routing module configured to determine a path of data to an appropriate target along a shortest path and at a low bandwidth;
a data module configured to store and process data to function as a cache server;
a security module configured to apply encryption computation to guarantee security of data; and
a network module configured to manage incoming and outgoing data and processing heterogeneous protocols.

3. The integrated predictive analytics apparatus of claim 2, wherein the network module comprises:
a net end-point module configured to provide various standard protocols to perform communication through the heterogeneous protocols; and
an abstract protocol configured to provide a common protocol to support various requests and responses from the net end-point module.

4. The integrated predictive analytics apparatus of claim 1, wherein the healthcare analytics unit is further configured to classify, recognize, and analyze the health data and match the health data to a corresponding disease template to generate health recommendation information.

5. The integrated predictive analytics apparatus of claim 1, wherein the health data repository stores a disease template, and
the healthcare analytics unit comprises:
an analytics and prediction module configured to obtain disease recognition data from the health data by using disease data stored in the health data repository;
a health recommendation system configured to generate the health recommendation information based on the disease recognition data; and
a cognitive health visualization module configured to visualize the health data.

6. The integrated predictive analytics apparatus of claim 1, wherein the healthcare analytics unit comprises a cognitive health visualization module configured to visualize the health data in a two-dimensional (2D) or three-dimensional (3D) form to allow the health data to be used by a web browser, a mobile device, a virtual reality (VR) device, or an application.

7. The integrated predictive analytics apparatus of claim 1, wherein the computation engine comprises a deep learning computation engine module, and
the deep learning computation engine module is configured to classify the health data according to a disease based on a disease template stored in the health data repository.

8. The integrated predictive analytics apparatus of claim 1, wherein the healthcare analytics unit further comprises a display displaying a user interface (UI), and
the display displays a medical image in a 2D or 3D form by visualizing the health data and allows a user to insert annotation to and evaluate the medical image.

9. The integrated predictive analytics apparatus of claim 1, wherein the healthcare analytics unit is further configured to visualize the health data on a web browser, and
the user uses the web browser by using an address of a server of the healthcare analytics unit.

10. The integrated predictive analytics apparatus of claim 1, wherein the integrated predictive analytics apparatus is paired with a virtual reality (VR) device, and
virtualizes the health data in a 3D form through the VR device and interacts with a user of the VR device.

11. An operation method of an integrated predictive analytics apparatus comprising a healthcare analytics unit, the operation method comprising:
receiving health data generated based on sensor data obtained from sensor devices; and
processing and visualizing the health data by the healthcare analytics unit.

12. The operation method of claim 11, further comprising:
receiving, by a hub included in the integrated predictive analytics apparatus, the sensor data; and
generating the health data by reconstructing the sensor data in a specific form, and transmitting the health data,
wherein the generating and transmitting of the health data comprise:
converting a protocol form of at least a part of the sensor data; and
performing routing to determine a path of the health data to an appropriate target.

13. The operation method of claim 11, wherein the processing and visualizing of the health data comprise:
classifying, recognizing, and analyzing the health data; and
generating health recommendation information by matching the health data to a disease template.

14. The operation method of claim 11, wherein the processing and visualizing of the health data comprise visualizing the health data in a two-dimensional (2D) or three-dimensional (3D) form to allow the health data to be used by a web browser, a mobile device, a virtual reality (VR) device, or an application.

15. The operation method of claim 11, wherein the processing and visualizing of the health data comprise:
classifying the health data as at least one disease based on a previously stored disease template by using machine learning classification; and
generating health recommendation information corresponding to the health data based on the classified disease.

16. The operation method of claim 15, wherein the processing and visualizing of the health data further comprise:
visualizing at least one of the health recommendation information or the health data;
outputting visualized information to the user;
receiving feedback information about the output visualized information from the user; and
using the feedback information for next processing.

17. A computer program product comprising one or more non-transitory computer-readable recording media having recorded thereon a program for executing an operation method of an integrated predictive analytics apparatus comprising a healthcare analytics unit,
the operation method comprising:
receiving health data generated based on sensor data obtained from sensor devices; and
processing and visualizing the health data by the healthcare analytics unit.

18. The computer program product of claim 17, wherein the operation method comprises:
receiving, by a hub included in the integrated predictive analytics apparatus, the sensor data; and
generating the health data by reconstructing the sensor data in a specific form, and transmitting the health data, and
the generating and transmitting of the health data comprise:
converting a protocol form of at least a part of the sensor data; and
performing routing to determine a path of the health data to an appropriate target.

19. The computer program product of claim 17, wherein the processing and visualizing of the health data further comprise:
classifying the health data as at least one disease based on a previously stored disease template by using machine learning classification; and
generating health recommendation information corresponding to the health data based on the classified disease.

20. The computer program product of claim 19, wherein the processing and visualizing of the health data further comprise:
visualizing at least one of the health recommendation information or the health data;
outputting visualized information to the user;
receiving feedback information about the output visualized information from the user; and
using the feedback information for next processing.
